(19) United States
(12) Patent Application Publication
(10) Pub. No.: US 2023/0225494 A1
(43) Pub. Date: Jul. 20, 2023

(54) TEETH CLEANING SYSTEM AND METHOD OF USE

(71) Applicant: Grin Labs, Inc., Wilmington, DE (US)

(72) Inventors: Kenneth Brown, Rancho Palos Verdes, CA (US); Andreas Dierks, Dubai (AE); Hans Michaelsen, Dubai (AE); Daniel Kristoffersen, Dubai (AE)

(21) Appl. No.: 18/122,659

(22) Filed: Mar. 16, 2023

Related U.S. Application Data
(63) Continuation-in-part of application No. 18/183,057, filed on Mar. 13, 2023.
Application No. 18/183,057 is a continuation of application No. 16/919,978, filed on Jul. 2, 2020, now Pat. No. 11,633,036.
(60) Provisional application No. 62/942,409, filed on Dec. 2, 2019.

(51) Int. Cl. A46B 9/04; A46B 15/00; A61B 1/05

(52) U.S. Cl. CPC A46B 9/045; A46B 15/0022; A61B 1/05; A46B 2200/1066

(57) ABSTRACT

A teeth cleaning system, which provides improved coverage or brushing area. The system includes an actuated tray comprises brushing elements. The brushing elements may be positioned to engage the front, rear, and top surfaces of the upper and lower jaw teeth. The actuation may be configured to oscillate the tray in a lateral direction which may brush all the surfaces of teeth simultaneously. Some embodiments include a higher density of bristles which increases the effectiveness of brushing. In addition, aspects of the system reduce the time spent brushing teeth.

### PRIORITY CLAIM

This application claims priority to and is a continuation-in-part application of U.S. Pat. Application No. 18/183,057 that was filed on Mar. 13, 2023, which was filed as a continuation of U.S. Pat. Application No. 16/919,978 that was filed on Jul. 2, 2020. U.S. Pat. Application No. 16/919,978 claims benefit under 35 U.S.C. § 119(e) of U.S. Provisional Application having Ser. No. 62/942,409 filed Dec. 2, 2019. Both applications are hereby incorporated by reference herein in their entirety.

### FIELD

The subject disclosure relates to brushing, scrubbing, and general cleaning, and more particularly, to implements which concurrently brush plural tooth surfaces.

### BACKGROUND

The field of teeth cleaning includes a number of approaches to brushing teeth. In the very basic, conventional approach is to provide a manually operated single brush head which the user applies to a single tooth or two overlapping teeth at a time until the user moves on.

Other approaches use electric powered toothbrushes in response to the ineffective approach of manual toothbrushes. Some electric toothbrushes power a single head in either a rotary motion (where the brush head spins) or in a reciprocating motion. Still, the single brush head approach requires time to apply the brush to each tooth.

Some devices have proposed multiple toothbrush heads but are limited in their effectiveness because of choice in the range of motion which for some is, for example, along an axis into and out of the user’s mouth. As may be seen, this may be ineffective because the direction of brushing does not follow the front face of the front teeth, which are the teeth most visible to others. Still yet, the rear surface of the front teeth may likewise be generally missed by such an approach.

In some instances, conventional brushing devices lack enough bristles to adequately reach each tooth surface simultaneously.

As can be seen, there is a need for a device that improves the coverage of teeth and thereby reduces the amount of time it takes.

### SUMMARY

In one aspect of the disclosure, a teeth cleaning system is disclosed. The system comprises: an arcuate tray configured to receive a set of teeth, wherein the tray includes: an inner arcuate wall, an outer arcuate wall, a baffle positioned transversely across between an inner surface of the outer arcuate wall and an inner surface of the inner arcuate wall and defining an upper channel for receiving teeth of an upper jaw and defining a lower channel for receiving teeth of a lower jaw, and a plurality of bristles positioned in the upper channel and in the lower channel; a handle; a connector, wherein the arcuate tray is coupled to the handle by the connector; a motor coupled to the handle; and an oscillator coupled to the connector, wherein the oscillator is configured to move the arcuate tray side-to-side along the same plane as the baffle in response to operation of the motor.

In another aspect of the disclosure, a teeth cleaning system is disclosed which, comprises: an arcuate tray configured to receive a set of teeth, wherein the tray includes: an inner arcuate wall, an outer arcuate wall, a baffle positioned transversely across between an inner surface of the outer arcuate wall and an inner surface of the inner arcuate wall and defining an upper channel for receiving teeth of an upper jaw and defining a lower channel for receiving teeth of a lower jaw, and a plurality of bristles positioned in the upper channel and in the lower channel, wherein the plurality of bristles comprises a density of between 324 bristles per cm² -646 bristles per cm²; a handle; a connector, wherein the arcuate tray is coupled to the handle by the connector; and a motor coupled to the handle, wherein operation of the motor brushes the teeth of the upper jaw and brushes the teeth of the lower jaw.

In yet another aspect of the disclosure a teeth cleaning system is disclosed which, comprises: an arcuate tray configured to receive a set of teeth, wherein the tray includes: an inner arcuate wall, an outer arcuate wall, a baffle positioned transversely across between an inner surface of the outer arcuate wall and an inner surface of the inner arcuate wall and defining an upper channel for receiving teeth of an upper jaw and defining a lower channel for receiving teeth of a lower jaw, and a plurality of bristles positioned in the upper channel and in the lower channel, wherein the plurality of bristles comprise a density between 324 bristles per cm² - 646 bristles per cm², herein a top surface of the baffle and a bottom surface of the baffle, the inner surface of the inner arcuate wall, and the inner surface of the outer arcuate wall each include a plurality of receptacles and wherein the plurality of bristles are positioned in bundles in respective receptacles; a handle; a connector, wherein the arcuate tray is coupled to the handle by the connector; a motor coupled to the handle, wherein operation of the motor brushes the teeth of the upper jaw and brushes the teeth of the lower jaw; and an oscillator coupled to the connector, wherein the oscillator is configured to move the arcuate tray side-to-side along a same plane as the baffle in response to operation of the motor.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive..

### BRIEF DESCRIPTION OF THE DRAWINGS

FIG. 1 is a perspective view of a teeth cleaning system in accordance with an aspect of the subject technology.

FIG. 2 is a top perspective view of a tray piece of the system of FIG. 1 in accordance with an aspect of the subject technology.

FIG. 3 is a side perspective view of the tray of FIG. 2.

FIG. 4 is a top view of the tray of FIG. 2.

FIG. 5 is a side view of the tray of FIG. 2.

FIG. 6 is a side perspective view of the tray of FIG. 2 with the bristles removed.

FIG. 7 is a bottom view of the tray of FIG. 2 with bristles removed.

FIG. 8 is a connector side end view of the tray of FIG. 2.

FIG. 9 is a perspective view of a motorized handle of the system of FIG. 1 in accordance with an aspect of the subject technology.

FIG. 10 is a front view of the handle of FIG. 9.

FIG. 11 is a side view of the handle of FIG. 9.

FIG. 12 is a bottom perspective view of the handle of FIG. 9.

FIG. 13 is a perspective, internal view of motorized elements in the handle of FIG. 9 in accordance with an aspect of the subject technology.

FIG. 14 is a front view of the handle of FIG. 13.

FIG. 15 is a partial front view of the handle of FIG. 13 with an enlarged view of an oscillator system and depicting a side-to-side oscillating motion of a shaft in accordance with an embodiment.

FIG. 16 is a front perspective view of the oscillator system of FIG. 15.

FIG. 17 is a bottom perspective view of a guide plate and shaft assembly in the oscillator system shown in FIG. 15.

FIG. 18 is a top perspective view of a cam system in the oscillator system of FIG. 15.

FIG. 19 is a top perspective view of a motor driving the oscillator system of FIG. 15 with the cam system removed.

FIG. 20 is a perspective, cross-sectional view of a cam interface in the oscillator system of FIG. 18 according to an exemplary embodiment.

FIG. 21 is an exploded, perspective, internal view of elements in the handle in accordance with an aspect of the subject technology.

FIG. 22 is a cross-sectional, perspective view of the elements in a handle of a teeth cleaning system in accordance with an aspect of the subject technology.

FIG. 23 is a cross-sectional, front view of the elements in the handle in accordance with an aspect of the subject technology.

FIG. 24 is a front view of a teeth cleaning system in accordance with an aspect of the subject technology.

FIG. 25 is a perspective view of the teeth cleaning system of FIG. 24 in accordance with an aspect of the subject technology.

FIG. 26 is a perspective view of the teeth cleaning system of FIG. 24 with an internal view of the handle assembly in accordance with an aspect of the subject technology.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. Like or similar components are labeled with identical element numbers for ease of understanding.

In general, exemplary embodiments of the subject technology provide a cleaning system for teeth that provides improved coverage area and an improved range of motion to more effectively brush teeth with motorized actuation. It will be appreciated that, aspects of the system include an arrangement of bristles that provide greater bristle density than in previous systems. The greater density covers more tooth surfaces to be brushed. The system includes a motor to automatically actuate the brushing features. The system may automatically brush all tooth surfaces during operation. Another aspect contributing to the improved brushing performance includes the motion of actuation used in some embodiments. The system may be configured to actuate the brushing in a side-to-side range of movement, which may be more natural to users.

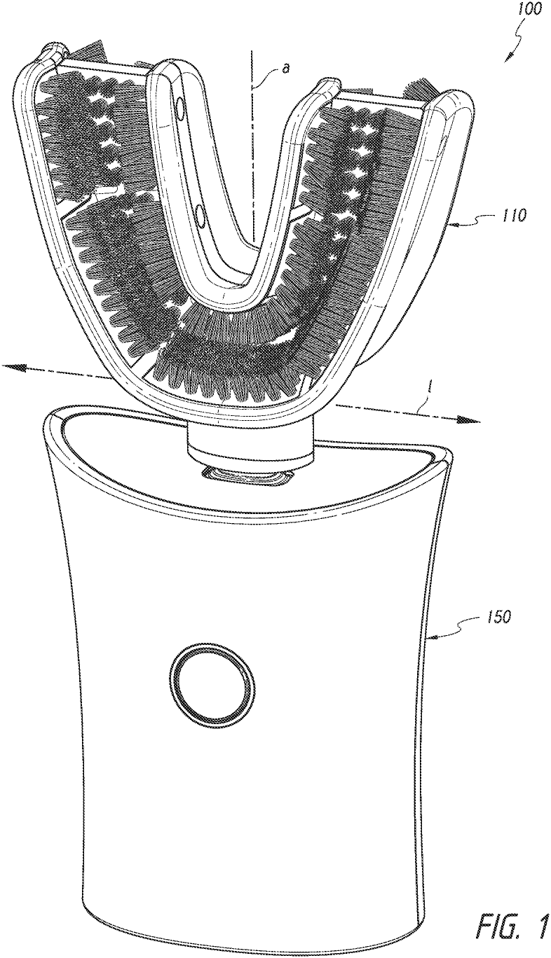

Referring now to FIG. 1, a teeth cleaning system 100 (referred to generally as the “system 100”) is shown according to an exemplary embodiment. The system 100 generally includes a tray assembly 110 (referred to sometimes as “tray 11 O”) and a handle assembly 150 (sometimes referred to as the “handle 150”). As will be described in further detail below, the handle assembly 150 is motorized. The tray 110 is connected to the handle 150 so that the tray 110 is actuated to brush a user’s teeth. In general use, the tray assembly 110 may be inserted into a user’s mouth (not shown) axially along an axis “a”. The axis “a″” represents the line or plane that extends from the bottom of the handle 150 to front of the tray assembly 110 and is generally in the direction of insertion. When the handle 150 is operated, the motor element (described below) may actuate the tray assembly 110 laterally (relative to the axis “a”) along an axis “/”. For example, the left and right sides of the tray 110 may move toward the left and right sides of the handle 150.

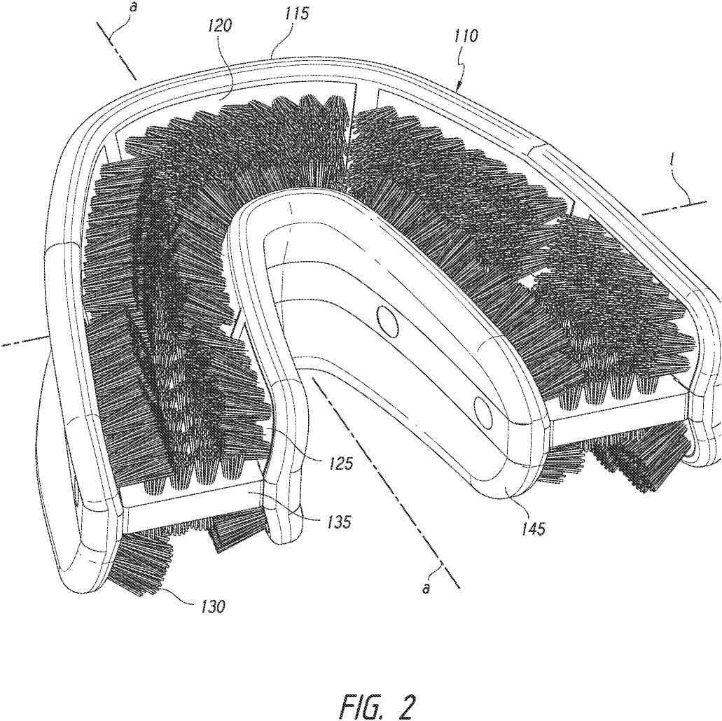

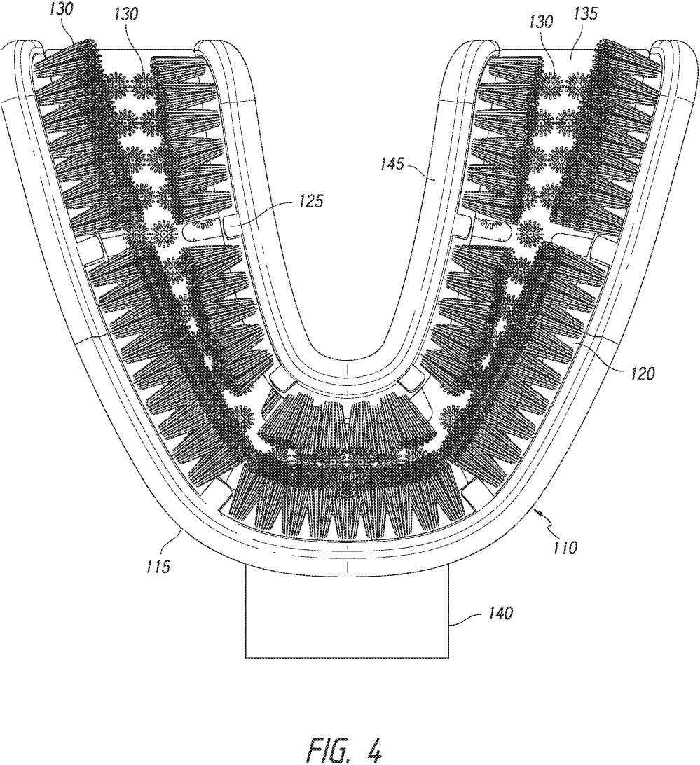

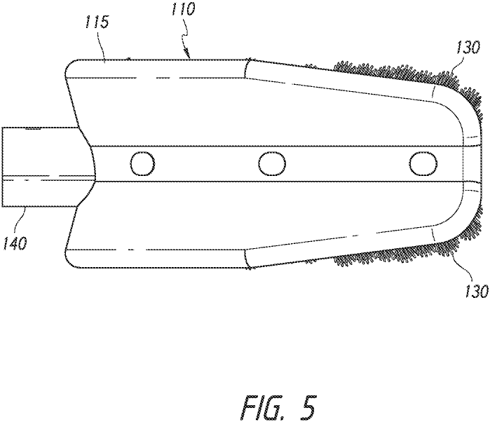

Referring now to FIGS. 2-8, the tray assembly 110 is shown according to an exemplary embodiment. The tray 110 may be generally arcuate to loosely index to the shape of a user’s jaw. In an exemplary embodiment, the tray 110 may include an outer arcuate wall 115 and an inner arcuate wall 145. In some embodiments, a baffle 135 may be positioned transversely to span across between an inner surface 120 of the outer arcuate wall 115 and an inner surface 125 of the inner arcuate wall 145. The baffle 135 may follow the general arcuate shape of the tray 110. In some embodiments, the baffle 135 may be parallel to the plane of the axis″/” and transverse to the axis “a” (see FIG. 2). The arrangement of the baffle 135 between the outer arcuate wall 115 and the inner arcuate wall 145 may define an upper arcuate channel for receiving teeth of an upper jaw and may define an arcuate lower channel for receiving teeth of a lower jaw in use. A plurality of brushing elements 130 is positioned in the channels of the tray assembly 110. The brushing elements 130 may be for example bristles (sometimes referred to as “bristle-hairs” or “strands”). In some embodiments, the bristles may be for example, nylon, microfiber, sponge, organic sponge, fabric, rubber, silicone, or bamboo.

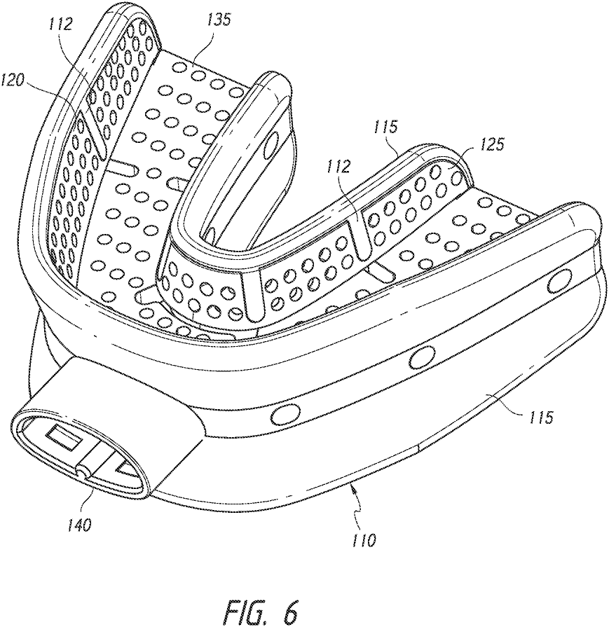

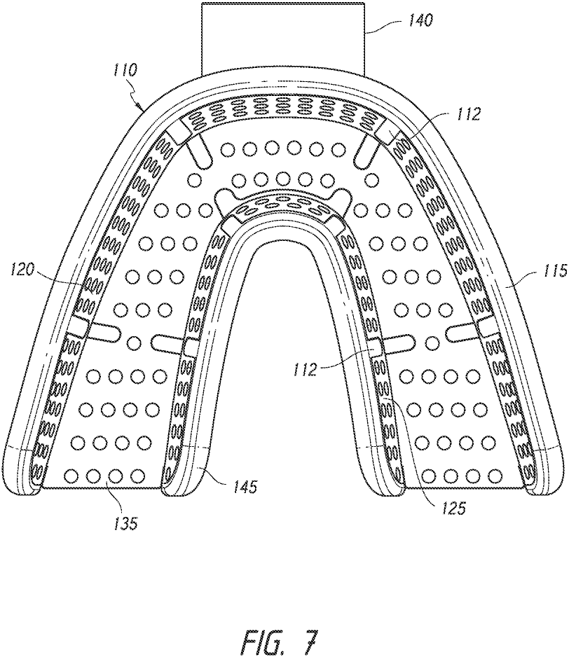

In some embodiments, the outer arcuate wall 115, the inner arcuate wall 145, and the top and bottom surfaces of the baffle 135 include a plurality of receptacles (holes) (FIGS. 6 and 7). Each of the receptacles may be configured to hold a bundle of bristles 130. The free ends of bristles 130 may be arranged to contact the anterior, posterior, and top surfaces of each tooth (as well as reach in between teeth and gums). In some embodiments, the bundles of bristles projecting from the inner surface 120 of the outer arcuate wall 115 and from the inner surface 125 of the inner arcuate wall 145 may project at an acute angle relative to the plane of the upper/lower surface of the baffle 135. In operation, as the tray 100 is actuated, the bristles 130 on the baffle 135 brush the top surfaces of teeth. The bristles on the inner surface 120 of the outer arcuate wall 115 and from the inner surface 125 of the inner arcuate wall 145 brush the anterior and posterior surfaces of teeth. The angle of the bristles on the inner surface 120 of the outer arcuate wall 115 and from the inner surface 125 of the inner arcuate wall 145 promote brushing along the height of a tooth’s anterior/posterior face. In some embodiments, the bristles 130 on the innermost and outermost rows of the baffle 135 may partially overlap with the bristles 130 (on the rows closest to the baffle 135) on the inner surface 120 of the outer arcuate wall 115 and of the inner surface 125 of the inner arcuate wall 145.

In an exemplary embodiment, the bristles may be arranged in a density of approximately 42 bristles per hole and 452.4 bristle-hairs per square centimeter. Embodiments may vary the bristle density between 324 bristles per cm² - 646 bristles per cm². As will be understood, the density of bristles may vary depending on factors such as bristles per hole, number of holes, spacing of holes, diameter of holes, and diameter of bristle strands used. For example, in embodiments that use softer bristles with thinner strand diameters than the embodiment with 42 bristles per hole, there may be approximately 84 bristles per hole which yields approximately the same density but with a softer brush rating. Similarly, embodiments using firmer bristles with thicker strand diameters may use 24 bristles per hole to achieve the same density. Accordingly, the bristle density may be manipulated by increasing or decreasing the strand diameter and/or the number of strands per hole. In addition, the other aforementioned factors (relating to hole position, size, and spacing) may be manipulated to generate a bristle density within the disclosed range. For example, more empty areas between holes may require more bristles to provide the same density and vice versa.

Thus, when referring to “bristle density”, the subject disclosure encompasses in general the density of bristle material per area with the understanding that substitutions may be made in any of the underlying factors of bristle density while still falling within the scope of the technology herein. The bristle material per area may be based on the diameter of a bristle used in an embodiment multiplied by the numbers of bristles per unit of area.

Embodiments may include 327 to 653 holes overall on the surfaces 120 and 125, and on both sides of the baffle 135. In an exemplary embodiment, there may be approximately 480 holes overall on the surfaces 120 and 125, and on both sides of the baffle 135. Embodiments may position holes with a distance of 1.8 mm to about 0.75 mm between holes. In some embodiments, there may be approximately 7.72 holes per cm² of a surface (120, 125, 135) to approximately 15.38 holes per cm². In an exemplary embodiment, there are approximately 11.31 holes per cm².

There may be approximately 42 bent bristles/hole. Embodiments may include a range of approximately 25-55 bristles per hole. The bristle thickness may range from 0.01 mm to 0.2 mm. For embodiments with 42 bristles per hole, the bristle thickness may be approximately 0.15 mm. In total, there may be approximately 13,100 to 26,120 bent bristle hairs inserted into the tray assembly 110. In an exemplary embodiment, there may be 19,200 bent bristle hairs inserted into the tray assembly 110. As will be seen, the density of bristles is above the conventional density of bristles in previous systems, which leads to improved coverage of the brushing area for the surfaces of teeth. With a minimal range of motion, the system 100 is able to brush teeth more efficiently. It will be appreciated that this is a significant improvement in technology since the range of space available to move laterally within a user’s mouth is limited. The density of bristles 130 in the subject technology allows for better brushing effectiveness in the limited range of lateral movement.

In some embodiments, the inner surface 120 of the outer arcuate wall 115 and/or the inner surface 125 of the inner arcuate wall 145 may include slots. See FIGS. 6 and 7. As will be appreciated, the slots 112 provide flexure in the tray 110 so that the tray 100 may flex to accommodate the fit of different sized jaws. In some embodiments, the outer arcuate wall 115 and the inner arcuate wall 145 may comprise shells of a soft plastic, for example, thermoplastic polyurethane, silicone, or a thermoplastic elastomer, providing support for holding the inner surfaces 120 and 125. In some embodiments, the inner surfaces 120 and 125 may comprise a material that is firmer than the outer arcuate wall 115, for example, a hard plastic (which may be for example, polypropylene, acrylonitrile butadiene styrene, or a polycarbonate plastic). As may be appreciated, the softer material of the outer arcuate wall 115 and the inner arcuate wall 145 provide a user comfort since these elements may contact the user’s inner cheek surfaces as the brushing elements 130 engage the user’s teeth and gums. In some embodiments, the inner surfaces 120 and 125 may be firm providing a solid support structure for detaining the brushing elements 130 in place during use.

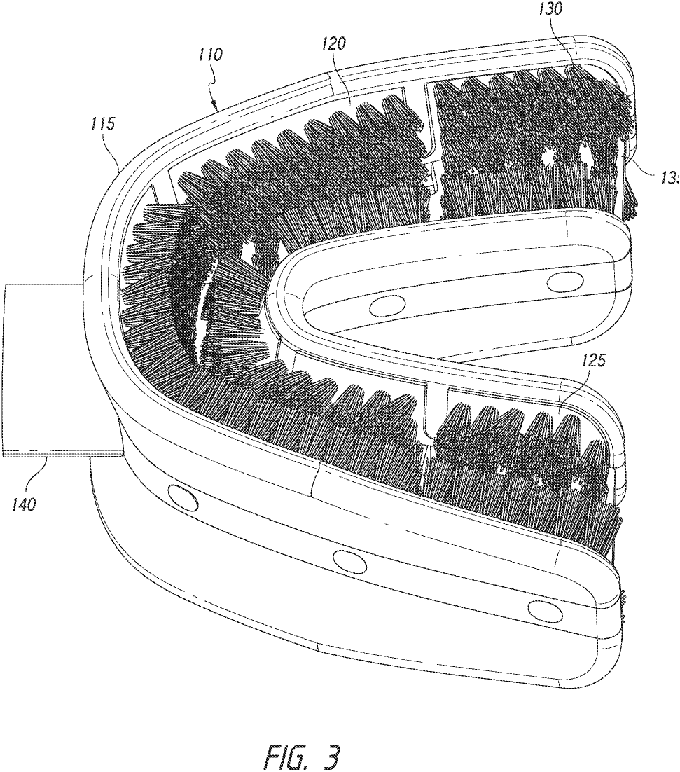

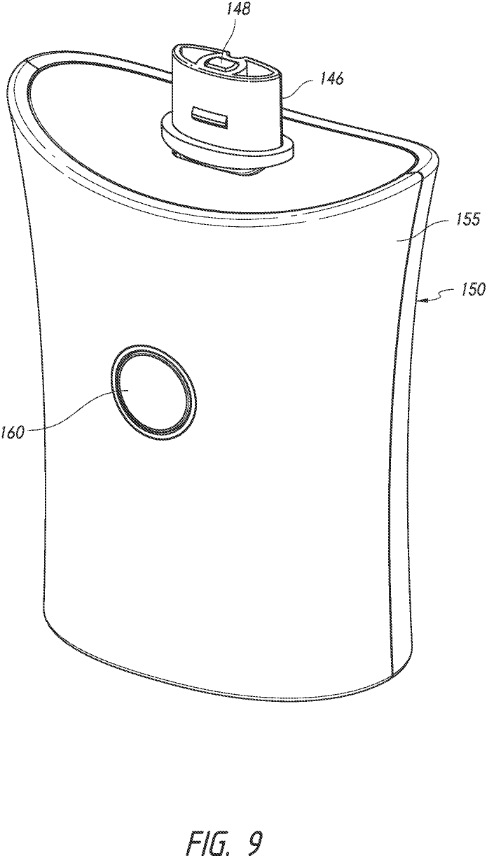

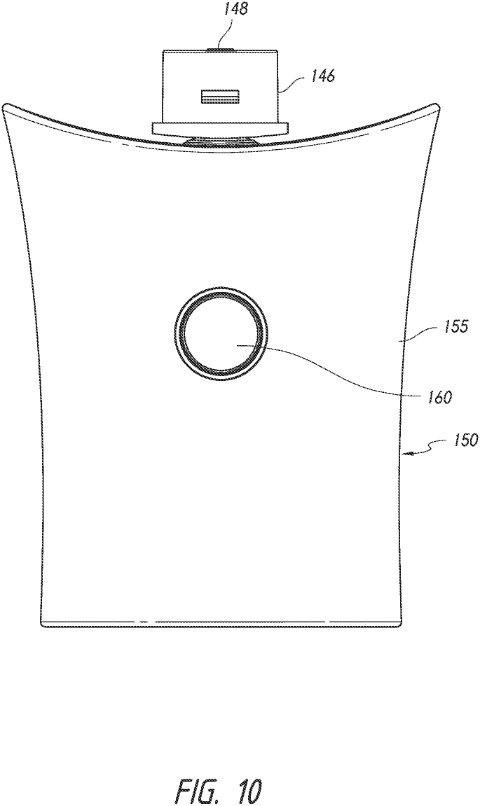

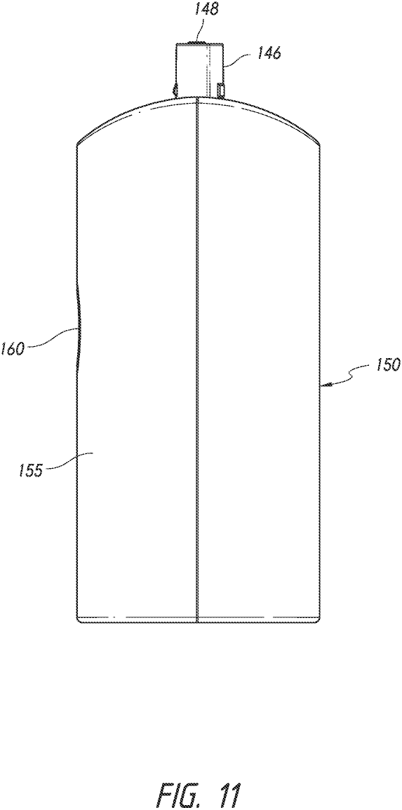

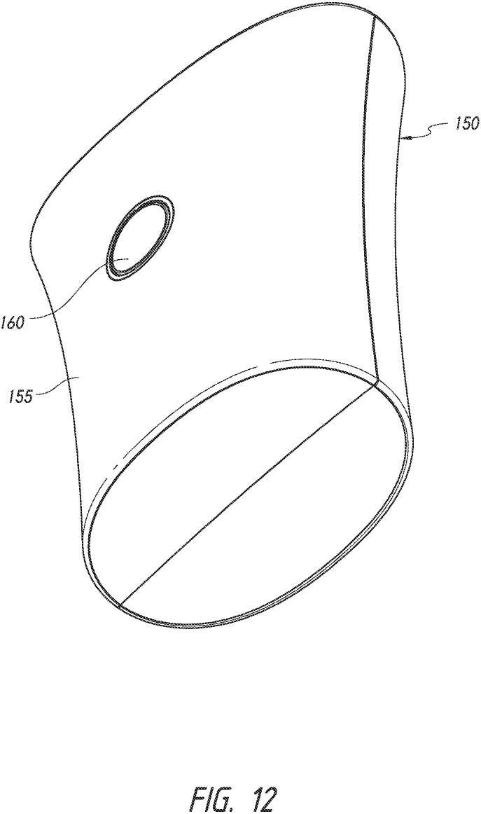

Referring now to FIGS. 3-8, in some embodiments, the tray assembly 100 may include a connector 140 coupled to an exterior surface of the outer arcuate wall 115. The connector 140 may be configured to receive the oscillator mechanism discussed below in more detail. For example, some embodiments may include a mating element 142 (for example, a female receptacle) which may receive a drive element (described below in reference to FIGS. 9-12) that actuates the tray assembly 110.

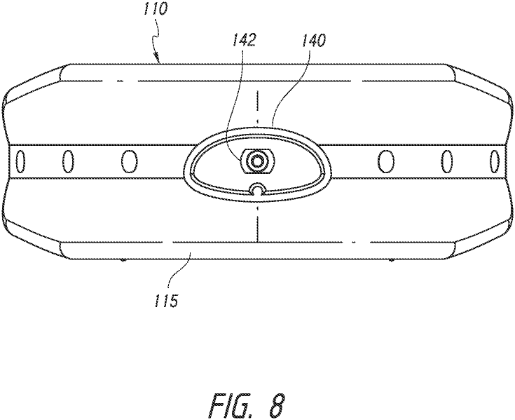

Referring now to FIGS. 9-12, the handle assembly 150 is shown according to an exemplary embodiment. The handle 150 includes a casing 155 and a button 160 which when triggered, activates the actuation of the tray assembly 110. The handle 150 may house an oscillator assembly 170. In an exemplary embodiment, the oscillator assembly 170 includes a shaft 172 (seen in FIG. 13) protruding from a top end of the casing 155. The shaft 172 may be covered by a sleeve 146. The sleeve 146 may be a male connection configured to slide into the opening in the connector 140. A distal end 148 of the shaft 172 may be configured to mate with the mating element 142 in the connector 140 shown in FIG. 8. The oscillator assembly 170 may be configured to move the shaft 172 side-to-side, which when the shaft 172 is actuated, drives the tray assembly 110 to oscillate from one side to the other in a lateral direction.

While the above mating configuration was described with the sleeve 146 attached to the handle 150, it should be understood that in other embodiments, the sleeve 146 may instead be in the connector 140 and the end 148 of the shaft 172 is inserted through the interior of the sleeve 146 to connect to the mating element 142.

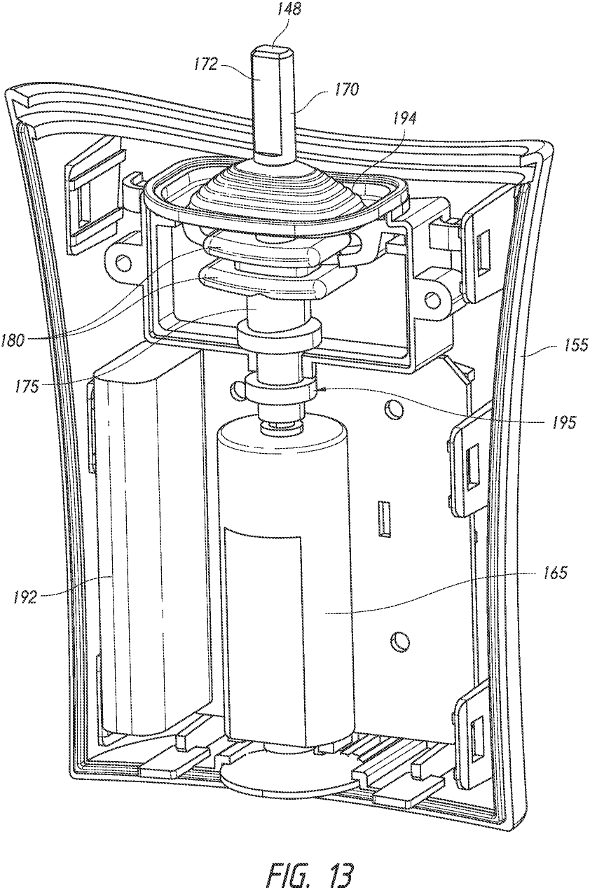

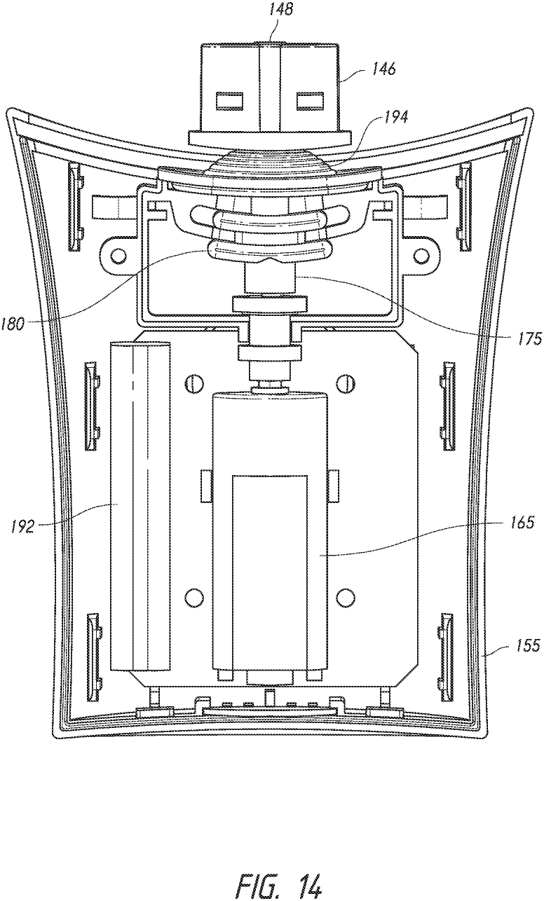

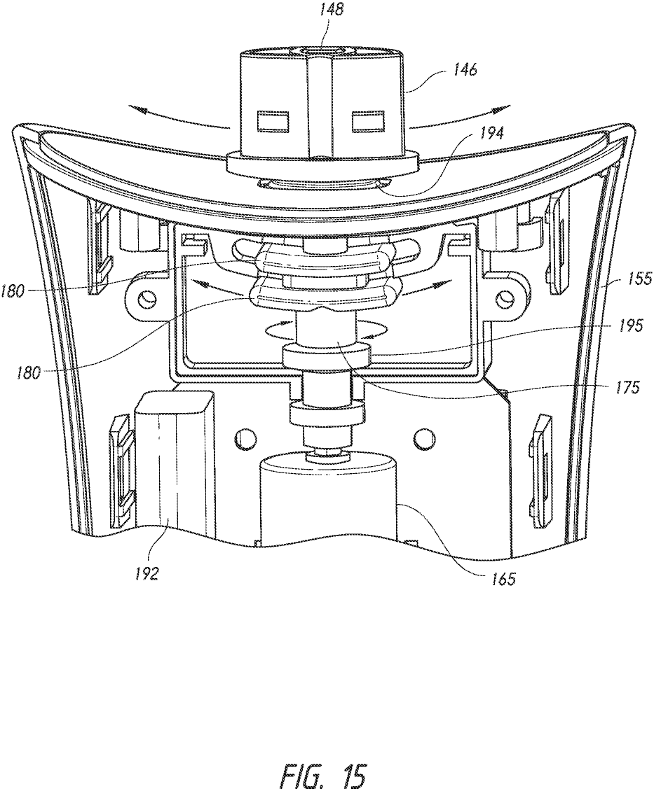

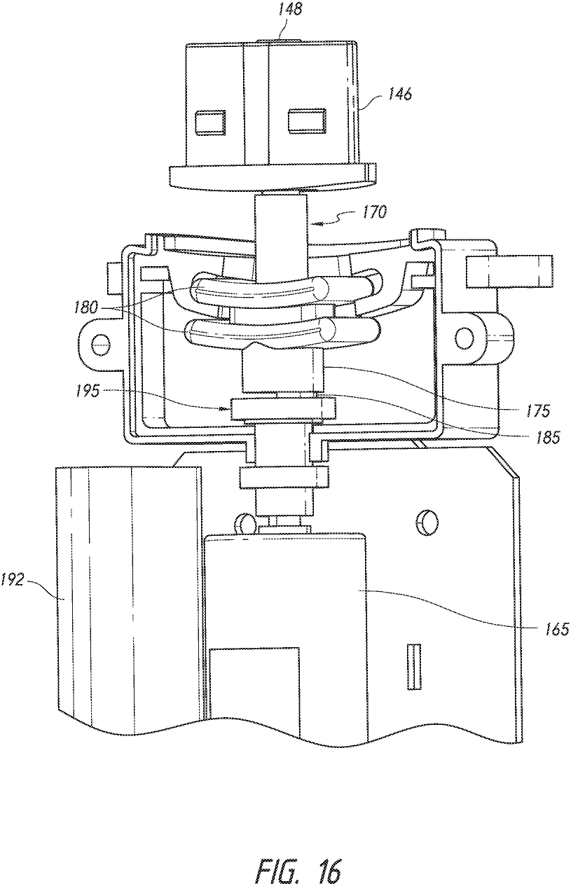

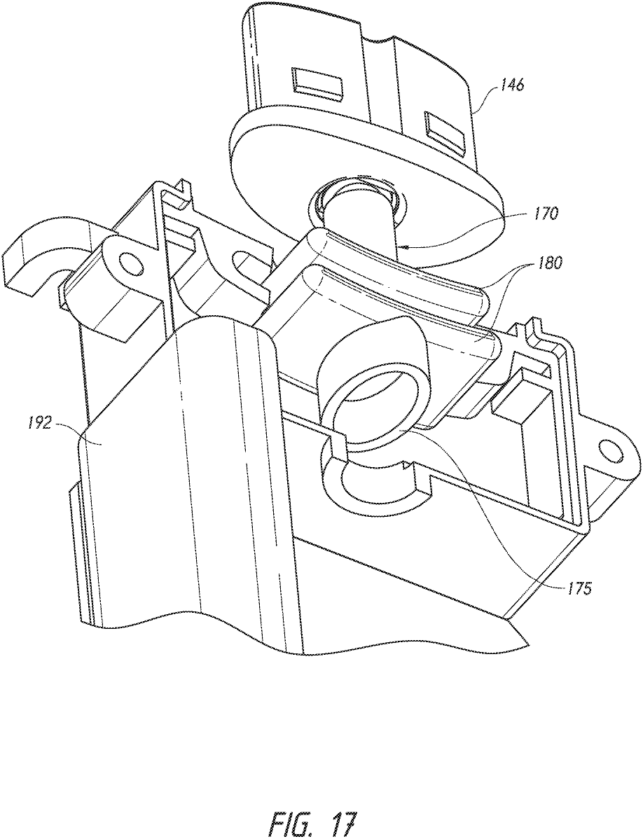

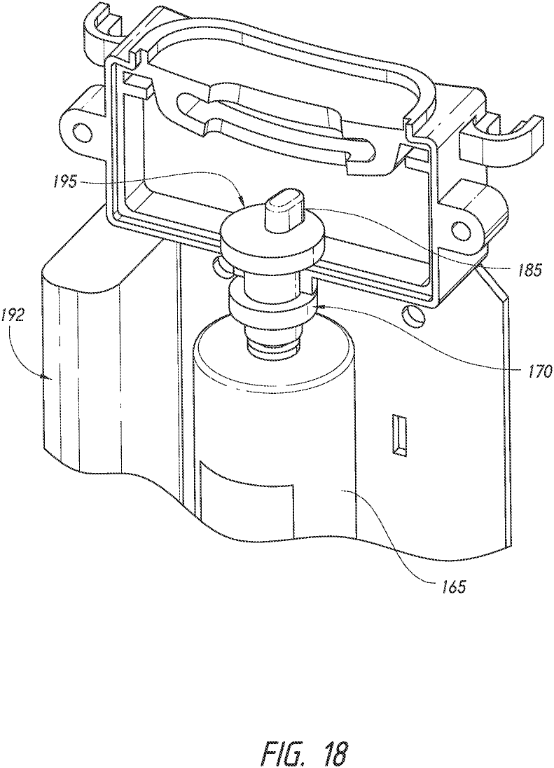

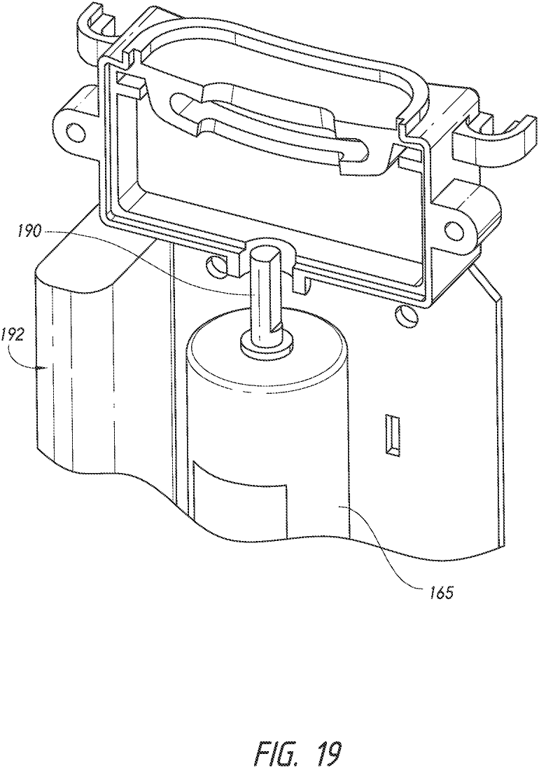

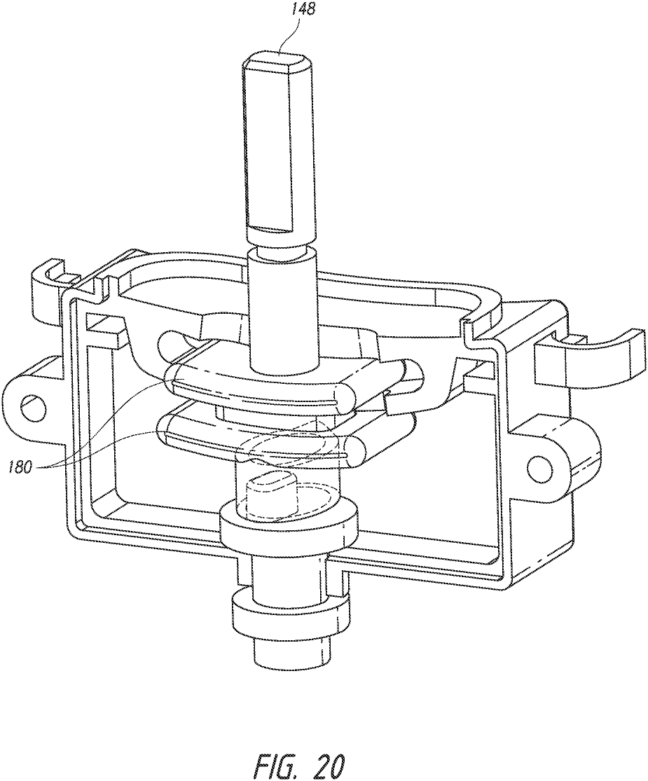

Referring now to FIGS. 13-20, the oscillator assembly 170 and other driving elements inside the casing 155 are shown according to an exemplary embodiment. The actuation from the handle 150 may be driven by a power source 192 which may be a battery, or a storage cell connected to a wall outlet source for plugged-in embodiments of the system 100. The oscillator assembly 170 may be driven by a motor 165. The motor 165 may include a drive shaft 190 (FIG. 19) which rotates during operation. The drive shaft 190 may be coupled to a cam assembly 195. The top of the cam assembly 195 may include a cam 185 (FIG. 18). The cam 185 may couple to a proximal end of the shaft 172. The cam 185 may be configured to, when engaged with the shaft 172, drive the shaft 172 in the lateral direction when the motor’s drive shaft 190 rotates the cam assembly 195. In some embodiments, the oscillator assembly 170 may include a sleeve 175 which houses a section of the shaft 172. As shown in FIG. 20, in an exemplary embodiment, the cam 185 may comprise a projection which is received in a pocket of the sleeve 175. As the cam 185 rotates, the projection causes the sleeve 175 to move laterally following the path of the cam rotation. The shaft 172 moves laterally with the sleeve 175. In some embodiments, the handle 150 may include one or more guide plates 180. The guide plates 180 may include slots through which the shaft 172 passes through. The guide plate 180 slot( s) may include a width that defines a range of movement that limits the travel of the shaft 172 in the lateral direction. The guide plates 180 ensure that the shaft 172 stays within a restricted path of motion. Some embodiments may include a cover 194 which prevents water from entering the handle and contacting the internal elements.

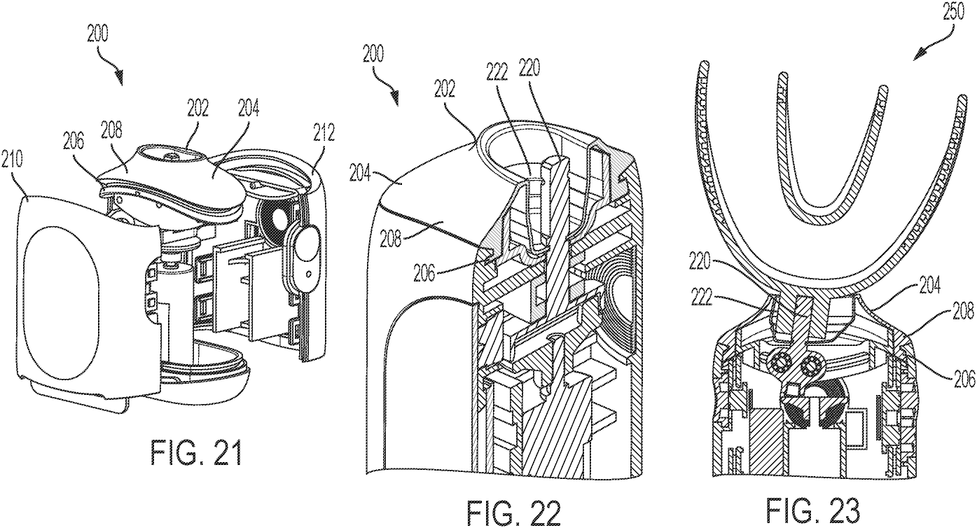

Referring now to FIGS. 21-23, an alternative embodiment of the teeth cleaning system is shown with a focus on the connection between a tray and the handle assembly. Certain embodiments may be water resistant with respect to the tray and the handle assembly. Reference numeral 200 represents a handle assembly 200 and reference numeral 250 represents the handle assembly 200 when it is connected to a tray. Handle assembly 200 is not connected to a tray in FIGS. 21-22. A flexible collar 204 may be used to protect the handle assembly 200 from water. The handle assembly 200 may include a front panel 210 and a back panel 212. As shown in FIGS. 21-23, the front panel 210 and the back panel 212 have recessed edges that fit into the flexible collar 204 when constructed. The flexible collar 204 may include an outer rim 202 at the upper portion of the flexible collar 204 and a void space 222 on the inside of the outer rim 202. The flexible collar 204 may also include an upper groove 208 and a lower groove 206 to form a tight fit with the corresponding portions of the handle assembly 200 (front panel 210, back panel 212). The upper groove 208 and lower groove 206 may assist in preventing any water from getting into the handle assembly 200. A male connector 220 from the handle assembly 200 may be configured to connect to the tray. The inner surface of this void space 222 of the flexible collar 202 fits tightly with the male connector 220 from the handle assembly 200 to prevent water from entering the handle assembly 200. Recessed portions of the male connector 220 enable connection with the flexible collar 204. The upper groove 208, the lower groove 206, and the inner surface of the void space 222 assist in preventing water from the tray or from use of the teeth cleaning system to enter the handle assembly 200.

The flexible collar 204 may be made out of silicone or TPU for sturdiness, but also flexibility. As discussed herein, the male connector 220 and the trey may move back and forth in operation. The flexible collar 204 is designed to flexibly move back and forth with the male connector 220 and the trey to keep the watertight fit with the handle assembly 200 and prevent water from entering the handle assembly 200. FIG. 23 shows how the flexible collar 204 may move in conjunction with both the male connector 220 and the connected trey. The void space 222 is designed to allow this motion of the trey and male connector 220 within this cavity of the flexible collar 204. As the trey moves right and left, the flexible collar 204 adjusts accordingly without breaking the seal.

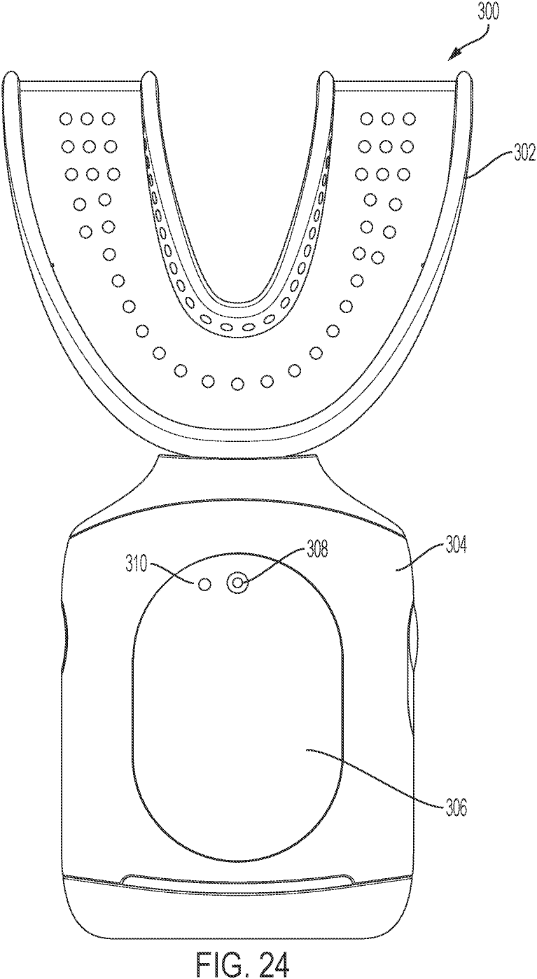

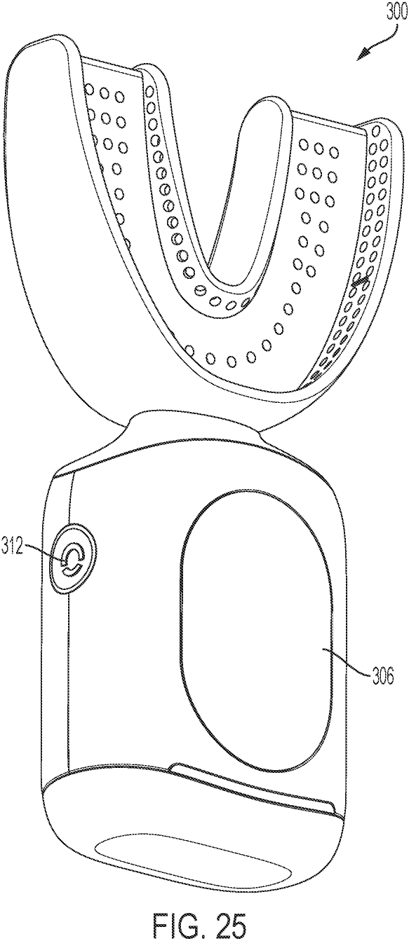

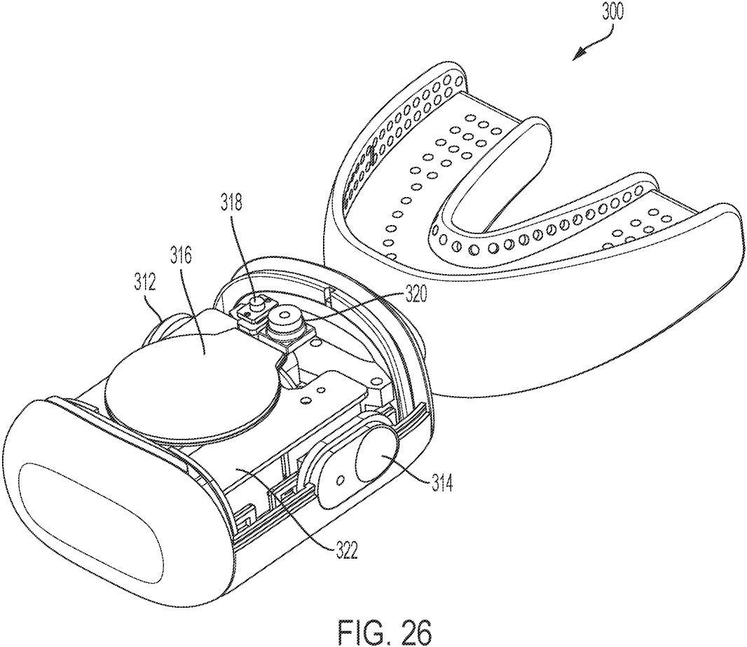

Referring now to FIGS. 24-26, an alternative embodiment of a teeth cleaning system 300 is shown with a display screen 316 (a cover for the display screen 306 is shown in FIG. 24) on a handle assembly 304. While this handle assembly 304 is shaped differently than handle assembly 150, it is designed to control a tray piece 302 as discussed above. However, the display screen 306 enables the teeth cleaning system 300 to provide the user with information, including a timer for brushing, the time of day, the weather outside, and other types of information. One of the most important functions of the display screen 306 may be to provide the user feedback when he or she is interacting with the device (e.g., directions on teeth cleaning, directions on taking images of the mouth or teeth). An audio speaker (not shown) may be used to provide audio feedback to the user. Connection to an internal network (wifi, cell network) of the user may be used to enable these features. Thus, a transceiver (not shown), which may be located on a printed circuit board (PCB), may be used to enable connection to this internal network or even external networks. Additionally, the cover of a display screen 306 in conjunction with a hole in the cover for a flash 310 and a second hole in the cover for a camera 308 enables the user to take images and video of the teeth, gums, and mouth of the user. A button 312, which may be located on either side of the handle assembly 304 may enable the user to turn on and off the handle assembly 304 and the corresponding display screen 316. An outer case and a cover for the display screen 306 have been removed in FIG. 26 to show the internal components of the handle assembly 150. More specifically, a flash 318 and a camera 320 provide the ability to take images and videos of the teeth, gums, face, and mouth of the user. A printed circuit board (PCB) 322 exists underneath the display screen 306 to house the hardware, firmware, and software necessary for features discussed herein. Two secondary buttons 314 may exist to control other features of the display screen 316, camera 320, flash and/or handle assembly 304. The hardware, firmware, and software of the teeth cleaning system 300 enable the features discussed below. Simple images of the user’s teeth and gums may be taken by the user for internal analysis by the teeth cleaning system 300 or for external analysis by external software or dental professionals. The transceiver (not shown) may be used to enable this connection to external software or dental professionals. The transceiver may consist of a transmitter and a receiver for transmitting and receiving data.

This camera 320, flash 318, and hardware/software may provide simple scan or dual scan capabilities according to certain embodiments. A machine learning component may exist on the PCB 322 to enable automatic capturing of simple scans for routine oral and dental photography. These images may be processed internally by the teeth cleaning system 300 or may be transmitted to a remote server for analysis by external software and/or dental professionals. A transmitter or transceiver may be used to transmit said data to an external network. A deep scan option may also be available for more in-depth dental imaging. This imaging and processing may enable the detection of dental plaque, tooth decay, gingivitis, periodontitis, gum recession, tooth surface shade, and demineralization. In some embodiments, in-depth dental imaging may also detect misplacement of the teeth, oral cancer, and other health-related symptoms, including exhaustion, depression, pulse, blood pressure, etc. It can be difficult for a user to keep track of his or her dental health and detect inconsistencies when they arise, especially if the user does not or cannot visit a dental professional regularly. These features can offer effortless oral health monitoring on a consistent basis, allowing users to detect any symptoms in the early stages before they evolve into serious health issues. The teeth cleaning device may also include a lift to wake sensor (not shown) that enables the features of the handle assembly 200 after the user lifts the device. This sensor may be controlled by the hardware, firmware, and software on the PCB.

In some embodiments, the hardware and software of the teeth cleaning system 300 uses machine learning technology to automatically detect a person’s face and teeth to make the process of capturing the necessary images less time consuming and cumbersome. An initial scan of the user’s teeth, gums, mouth, and face may be necessary to set a baseline for this type of machine learning technology. Data storage may be required to store said images and deep scans of said images. Directions or instructions for taking the scans, images, or videos of the user’s teeth and gums may be given by the teeth cleaning device 300 through a speaker (not shown) or by instructions on the display screen 316. The directions may also be given through an app on a user’s mobile device that communicates with the teeth cleaning system 300 through a network. These directions may include commands concerning placement, movement, or handling of the teeth cleaning system 300 while the images or scans are being taken. More specifically, the directions for the scanning may be sent from an external server or cloud network and may be triggered by the user’s input in the app on the user’s mobile device. In some embodiments, complex instructions may be required to ensure that the teeth cleaning system 300 can detect the user’s face, smile, and teeth. The images or scans taken by the teeth cleaning system 300 may be updated or annotated by the user, software, or dental professional in certain embodiments. Segmentation and/or cropping out may also be necessary to process and analyze the user’s teeth and gums. A user’s interaction with the app on a mobile phone may enable additional features of the teeth cleaning system, including tracking of dental activities, dental appointments, videos of dental care, dental care tips, etc.

The flash 318 may be covered with a type of Fresnel lens to light up the teeth as well as autofocus technology that improves the quality of the images or scans taken of the user’s teeth and gums. The camera 320 may be a 5-mega pixel camera that offers colored images or could also be an IR camera (LiDAR). The hardware and software (at least partially on the PCB 322) ensures that the data from the corresponding images or scans may be acquired, processed, and sent to the external server or cloud network according to a secure protocol to protect the user’s health data. As discussed above, these images and scans may be part of a broader oral care system where teledentistry is available. The high-resolution images and scans may be accessed and analyzed by a dental professional, which may enable remote diagnostics and treatment. Additionally, the dental professional may utilize the benefit of machine learning technology for assistance in dental treatment as the catalog of dental images may provide a better overall view of the user’s dental health. Teledentistry may also be accomplished through the corresponding app on a mobile phone.

Those of skill in the art would appreciate that various components may be arranged differently (e.g., arranged in a different order, or partitioned in a different way) all without departing from the scope of the subject technology.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa, Headings and subheadings, if any, are used for convenience only and do not limit the invention.

Terms such as “top,” “bottom,” “front,” “rear,” “above,” “below,” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference. Similarly, an item disposed above another item may be located above or below the other item along a vertical, horizontal or diagonal direction; and an item disposed below another item may be located below or above the other item along a vertical, horizontal or diagonal direction.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

What is claimed is:

1. A teeth cleaning system comprising:
a handle assembly that is configured to be connected to a tray that is configured to be inserted into a user’s mouth;
a display screen within said handle assembly that is configured to be viewed by a user;
a flash within said handle assembly that is configured to emit light when activated;
a camera within said handle assembly that is configured to capture an image inside said user’s mouth; and
at least one processor that is configured to control said flash and said camera to capture a first set of images inside said user’s mouth and a second set of images inside said user’s mouth, and is configured to process said first set of images and said second set of images;
wherein said at least one processor is further configured to compare said first set of images of said user’s mouth to said second set of images.

2. The teeth cleaning system of claim 1, wherein said teeth cleaning system further comprises a transmitter for transmitting data related to said first set of images and said second set of images to an external network.

3. The teeth cleaning system of claim 2, wherein said external network is connected to an app of a mobile device.

4. The teeth cleaning system of claim 1, wherein said teeth cleaning system further comprises a receiver for receiving data from an app of a mobile device.

5. The teeth cleaning system of claim 3, wherein said data received from said app of said mobile device controls said flash and said camera to capture said images inside of said user’s mouth.

6. The teeth cleaning system of claim 4, wherein said data received from said app of said mobile device comprises information related to a position of said teeth cleaning system with respect to said mouth of said user.

7. The teeth cleaning system of claim 3, wherein said data received from said app of said mobile device controls said display screen that is configured to display information to said user.

8. The teeth cleaning system of claim 1, wherein said processor is further configured to compare said first set of images of said user’s mouth to said second set of images to assist in obtaining a deep scan of said user’s mouth.

9. The teeth cleaning system of claim 8, said teeth cleaning system further comprises a transmitter for transmitting data related to said deep scan of said user’s mouth to an external network.

10. The teeth cleaning system of claim 1 wherein said teeth cleaning system further comprises a memory for storage of data related to said deep scan of said user’s mouth.

11. The teeth cleaning system of claim 1, wherein said processor accesses said storage of data related to said deep scan of said user’s mouth to assist in obtaining a second deep scan of said user’s mouth.

12. A system for cleaning teeth comprising:
a handle assembly that is configured to be connected to a tray that is configured to be inserted into a user’s mouth;
a flash within said handle assembly that is configured to emit light when activated;
a camera within said handle assembly that is configured to capture an image inside said user’s mouth;
at least one processor that is configured to control said flash and said camera to capture a first set of images inside said user’s mouth and a second set of images inside said user’s mouth, and is configured to process said first set of images and said second set of images, wherein said at least one processor is further configured to compare said first set of images of said user’s mouth to said second set of images; and
at least one transceiver that is configured to transmit data related to said comparison of said first set of images and said second set of images to an external network.

13. The teeth cleaning system of claim 12, wherein said external network is connected to an app on a mobile device of said user.

14. The teeth cleaning system of claim 13, wherein said transceiver is configured for receiving data from an app of a mobile device.

15. The teeth cleaning system of claim 14, wherein said data received from said app of said mobile device controls said flash and said camera to capture said images inside of said user’s mouth.

16. The teeth cleaning system of claim 13, wherein data received from said app of said mobile device controls said display screen that is configured to display information to said user.

17. A teeth cleaning system comprising:
a handle assembly that is configured to be connected to a tray that is configured to be inserted into a user’s mouth and clean said user’s teeth;
a flash within said handle assembly that is configured to emit light when activated;
a camera within said handle assembly that is configured to capture an image inside said user’s mouth;
at least one processor that is configured to control said flash and said camera to capture a first set of images inside said user’s mouth and a second set of images inside said user’s mouth, and is configured to process said first set of images and said second set of images, wherein said at least one processor is further configured to compare said first set of images of said user’s mouth to said second set of images to assist in obtaining a deep scan of said user’s mouth; and
at least one transmitter that is configured to transmit data related to said deep scan of said user’s mouth to an external network.

18. The teeth cleaning system of claim 17, wherein said teeth cleaning system further comprises a memory for storage of data related to said deep scan of said user’s mouth.

19. The teeth cleaning system of claim 18, wherein said processor accesses said storage of data related to said deep scan of said user’s mouth to assist in obtaining a second deep scan of said user’s mouth.

20. The teeth cleaning system of claim 17, said teeth cleaning system further comprises a receiver for receiving data from an app of a mobile device.
